(19) United States
(12) Patent Application Publication
(10) Pub. No.: US 2023/0248659 A1
(43) Pub. Date: Aug. 10, 2023

(54) IMMUNE SUPPRESSING NANOPARTICLES FOR ROBUST SENSITIZATION OF DRUG-RESISTANT CANCER

(71) Applicant: King Abdulaziz City for Science and Technology ( KACST), Riyadh (SA)

(72) Inventors: Ali H. ALHASAN, Riyadh (SA); Haneen OMAR, Riyadh (SA); Roa' FARDOUS, Riyadh (SA); Yasser ALHINDI, Riyadh (SA); Abdulaziz ALMALIK, Riyadh (SA); Waleed M. ALGHAMDI

(21) Appl. No.: 17/969,587

(22) Filed: Oct. 19, 2022

Related U.S. Application Data
(60) Provisional application No. 63/257,187, filed on Oct. 19, 2021.

(51) Int. Cl. A61K 9/51; A61K 38/17; A61K 9/00; A61P 35/00

(52) U.S. Cl. CPC A61K 9/5161; A61K 9/0073; A61K 38/1741; A61P 35/00

(57) ABSTRACT

An immune-suppressing nanoparticle (NP) decorated with alpha-1 acid glycoprotein (AGP), an anti-inflammatory protein, circumventing the resistance of breast tumor cells to chemotherapy in addition to suppressing the tumor metastasis and invasion is disclosed. Methods of making hyaluronic acid-chitosan nanoparticles decorated with AGP (AGP-HA NPs) by sequential ionic gelation, spray drying, and AGP-surface adsorption are also disclosed. Treatment options to strengthen the anti-cancer effects of chemotherapeutic agents and potentially improve the survival rate of patients with metastatic breast cancer using the disclosed AGP-HA NPs containing agents are also disclosed.

### CROSS-REFERENCE TO RELATED APPLICATION

This application claims the benefit of priority to Provisional Application No. 63/257,187, filed Oct. 19, 2021, the entire contents of which are expressly incorporated herein by reference.

### FIELD OF INVENTION

The present disclosure generally relates to an immune-suppressing nanoparticles, such as hyaluronic acid-chitosan nanoparticles, decorated with alpha-1 acid glycoprotein (AGP) and at least one anti-inflammatory protein. The present disclosure also relates to methods of making such nanoparticles, as well as methods of treating cancer, including metastatic breast cancer, using such nanoparticles.

### BACKGROUND

Cancer treatment strategies, including surgery, chemotherapy, radiation therapy, and immunotherapy, have faced many clinical complexities over the last four decades affecting their therapeutic efficacy. Post-surgery chemotherapy is one of the most potent strategies for solid tumor cancer treatments; however, it poses tremendous challenges that lessen the therapeutic indices. One of the major impediments to chemotherapy treatment is the emergence of the multiple drug resistance (MDR) and metastatic characteristics, contributing to the failure of chemotherapies in variance cancers, including breast cancer. See e.g., Vyas, D., Laput, G. & Vyas, A. K., Onco Targets Ther 7, 1015-1023, doi:10.2147/OTT.S60114 (2014) and Gurunathan, S., Kang, M.-H., Qasim, M. & Kim, J.-H., Int J Mol Sci 19, 3264, doi:10.3390/ijms19103264 (2018), which are incorporated by reference for these specific teachings.

Multi-drug resistance (MDR) is a phenomenon where tumor cells develop resistance to chemotherapeutic drug molecules leading to limit their therapeutic efficacy. It has a complicated mechanism and can be mediated by various factors, greatly associated with drug efflux, mutation of oncogenes caused by previous treatments, and tumor cells adapting to altered microenvironment including epithelial-mesenchymal transition (EMT) process. Indeed, MDR in addition to metastatic and migratory characteristics of tumors are responsible for the failure of chemotherapy via inflammation-induced metastasis of hot tumors as a coping mechanism for survival. Hot tumors induces the activation of Nuclear Factor-κB (NF-κB), leading to the release of the cellular growth factor and the pro-inflammatory cytokine Tumor Necrosis Factor-alpha (TNF-α) and the production of the transforming growth factor TGF-β, promoting inflammation and cell survival; and mediating EMT and metastasis, respectively.

Intense research efforts have utilized nanoparticles (NPs) as one of the most promising interventions to overcome MDR. Previously utilized nanoparticle-based approaches have shown potential in manipulating MDR phenotype for drug uptake and retention, albeit rather ineffective in addressing the underlining inflamed tumors and the resultant metastasis. Recently, hyaluronic acid chitosan NPs (HA NPs) have been reported to exhibit a remarkable feature by adsorbing a unique anti-inflammatory protein alpha-1 acid glycoprotein (AGP) on their surface upon mixing with human serum proteins. HA has proven to be a natural selective CD44-targeting moiety that allows for targeting tumor cells bearing CD44 receptors, including breast cancer cells.

On the other hand, soluble AGP can suppress the overexpression of pro-inflammatory cytokines in the tumor microenvironment, including (TNF-α) and interleukins (IL-6) from tumor cells, in which they are linked to drug resistance and metastasis.

There is a need for an immune suppressing nanoparticles to validate the nano-platform in drug-resistant breast cancer cells. As described and claimed herein, the Inventors have devised an anti-inflammatory protein alpha-1 acid glycoprotein (AGP) decorated into HA NPs can be an alternative approach to modulate MDR via turning hot tumors into cold tumors and subsequently restoring their drug sensitivity and preventing their invasiveness. This innovative approach can be adopted to sensitize MDR phenotype and deliver low-dose chemotherapeutic agents to maximize efficacy while minimizing toxicity.

The disclosed nanoparticles are directed to overcoming one or more of the problems set forth above and/or other problems of the prior art

### SUMMARY

In one embodiment, there is disclosed a composition for reducing multi-drug resistance of tumor cells to chemotherapeutic drug molecules, the composition comprising: at least one nanoparticle comprising at least one anti-inflammatory protein, wherein the at least one anti-inflammatory protein comprises an alpha-1 acid glycoprotein (AGP), wherein the composition inhibits the proliferation and the migration of tumor cells by suppressing overexpressed pro-inflammatory cytokines. In one embodiment, the at least one nanoparticle comprises hyaluronic acid-chitosan.

In one embodiment, there is disclosed a method of making a composition comprising at least one nanoparticle comprising at least one anti-inflammatory protein, wherein the at least one anti-inflammatory protein comprises an alpha-1 acid glycoprotein (AGP), the method comprising sequential ionic gelation followed by at least one spray drying step. For example, the sequential ionic gelation steps comprise: preparing chitosan tripolyphosphate (CS/TPP) nanoparticles by using inter-molecular and intra-molecular ionic linkages created between negatively charged groups on the pentasodium tripolyphosphate (TPP), and positively charged amino groups of the chitosan. In this embodiment, the method may further comprising adding the CS-TPP nanoparticles to a hyaluronic acid solution to produce hyaluronic acid nanoparticles. In this embodiment, the hyaluronic acid nanoparticles maybe configured such that hydrogen bonding and electrostatic attractions occur between the carboxylic groups of hyaluronic acid and amine groups of chitosan.

In this disclosed method, the at least one spray drying step produces a powder comprising said nanoparticles, wherein the method may further comprising mixing the powder comprising said nanoparticles with AGP to produce hyaluronic acid-chitosan nanoparticles decorated with AGP.

In one embodiment, there is disclosed a method of treating cancer, comprising: treating cancer cells with a composition comprising at least one nanoparticle, such as hyaluronic acid-chitosan, comprising at least one anti-inflammatory protein, wherein the at least one anti-inflammatory protein comprises an alpha-1 acid glycoprotein (AGP); and treating the cancer cells with an anti-cancer, chemotherapeutic drug. Treating cancer cells with a composition comprising at least one nanoparticle comprising at least one anti-inflammatory protein, wherein the at least one anti-inflammatory protein comprises an alpha-1 acid glycoprotein (AGP) may be done prior to or simultaneous with treating said cancer cells with an anti-cancer, chemotherapeutic drug.

In one embodiment, the step of treating cancer cells with the disclosed composition comprises using an amount of the composition sufficient to sensitize the cancer cells prior to exposing the cancer cells to the anti-cancer, chemotherapeutic drug.

In one embodiment, the types of cancer that may be treated with the disclosed composition comprises metastatic cancer, such as metastatic breast cancer.

### BRIEF DESCRIPTION OF THE DRAWINGS

The accompanying drawings, which comprise a part of this specification, illustrate several embodiments and, together with the description, serve to explain the principles disclosed herein. The patent or application file contains at least one drawing executed in color. Copies of this patent or patent application publication with color drawing(s) will be provided by the Office upon request and payment of the necessary fee. In the drawings:

FIG. 1 is a scheme 1 showing the synthesis of AGP-HA NPs.

FIG. 1(a) are SEM images and their particle distribution and FIG. 1(b) DLS analysis of HA NPs.

FIG. 2(a) Zeta Potential and FIG. 2(b) FT-IR spectra of CS-TPP, HA NPs and AGP-HA NPs and their separate component.

FIG. 3(a) and FIG. 3(b) show wound healing of HA NPs, AGP, AGP-HA NPs at 24 and 48 hr incubation time mediate with LPS in MCF-7 cells, (c) Cytotoxicity of HA NPs at 24 and 48 hr incubation time using AlamarBlue assay in MCF-7 cells.

FIG. 4 shows LPS-concentration effect in the invasive potential of MDA-MB-231 cells at different LPS concentrations (5, 10 and 20 µg/ml) and different time intervals (0, 4, 12, 24 and 48 h).

FIG. 5 shows Cytotoxicity of AGP-HA NPs at 48 and 72 hr incubation time using a ccK-8 kit and LPS mediates DOX-resistance in MDA-MB-231 cells at various concentration of AGP-HA NPs (50, 100 and 300 µg/ml) at 48 and 72 hr incubation time.

FIG. 6 is an X-ray diffraction pattern of CS-TPP, HA NPs and their separate component.

FIG. 7 is a UV-VIS spectrum of free AGP, HA NPs and AGP-HA NPs.

FIG. 8 shows LPS-concentration effect in the invasive potential of MDA-MB-231 cells at different LPS concentrations (5, 10 and 20 µg/ml) at 0 and 24 h time intervals imaged using Cytation 5™ multi-mode Microplate Reader-Gen5™ software.

### DETAILED DESCRIPTION

By way of overview, there is described an immune-suppressing nanoparticle (NP) decorated with alpha-1 acid glycoprotein (AGP), an anti-inflammatory protein, circumventing the resistance of breast tumor cells to chemotherapy in addition to suppressing the tumor metastasis and invasion. Methods of making hyaluronic acid-chitosan nanoparticles decorated with AGP (AGP-HA NPs) by sequential ionic gelation, spray drying, and AGP-surface adsorption are also disclosed. The morphology, size distribution, surface charge density, and composition of the synthesized NPs were characterized to verify the successful interaction between the NPs and AGP. The efficacy of AGP-HA NPs to sensitize cells with MDR phenotypes as a function of enhancing the potency of free anti-cancer drug doxorubicin (DOX), where it is considered to be the most effective chemotherapeutic agent in breast cancer treatment. Synergistic antitumor effects between HA and AGP as evidenced by the simultaneous inhibition of tumor proliferation and migration, and the significantly enhanced efficacy of DOX post sensitizing MDA-MB-231 is also disclosed. Treatment options to strengthen the anti-cancer effects of chemotherapeutic agents and potentially improve the survival rate of patients with metastatic breast cancer using the disclosed AGP-HA NPs containing agents are also disclosed.

In one embodiment, HA NPs were prepared by an ionic gelation method. In this method, CS-TPP NPs were prepared based on the inter- and intra-molecular ionic linkages created between the negatively charged groups of pentasodium tripolyphosphate (TPP), and the positively charged amino groups of CS. Consequently, CS-TPP NPs were added to the HA solution to yield HA NPs in which hydrogen bonding and electrostatic attractions took place between the carboxylic groups of HA and the amine groups of CS. Next, the produced NPs were dried using the nano-spray drying technique, to yield a powder NPs with a large amount and better redispersion. Eventually, the dried NPs were mixed with AGP solution to yield AGP-HA NPs.

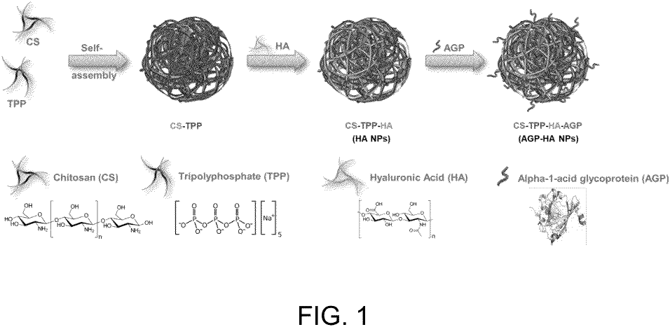

The size and the morphology of the HA NPs were assessed via scanning electron microscopy (SEM) (FIG. 1), revealing spherically shaped NPs with size distribution between 200 and 500 nm with a dominant particle size of 320 nm. Dynamic light scattering (DLS) measurement displayed a single population of NPs having an average hydrodynamic diameter of 352 ±22 nm (FIG. 1b).

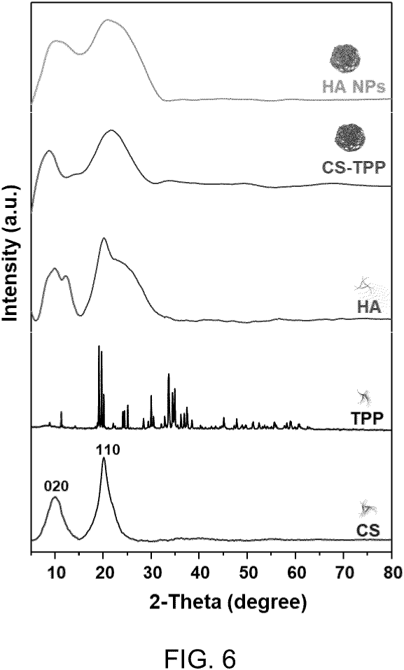

The X-ray diffraction (XRD) patterns of CS displayed two distinct diffraction peaks at 2θ = 10° and 20° corresponding to the crystallographic planes (020) and (110) as shown in FIG. 6. After TPP addition, the XRD peaks of CS were destroyed and reduced due to the crosslinking with TPP to form CS-TPP NPs. In HA NPs, the XRD peaks were enhanced in comparison to CS-TPP NPs owing to the interactions between amino groups of CS with carboxyl groups of HA, which limited the movement of CS chain and reduced its crystallization.

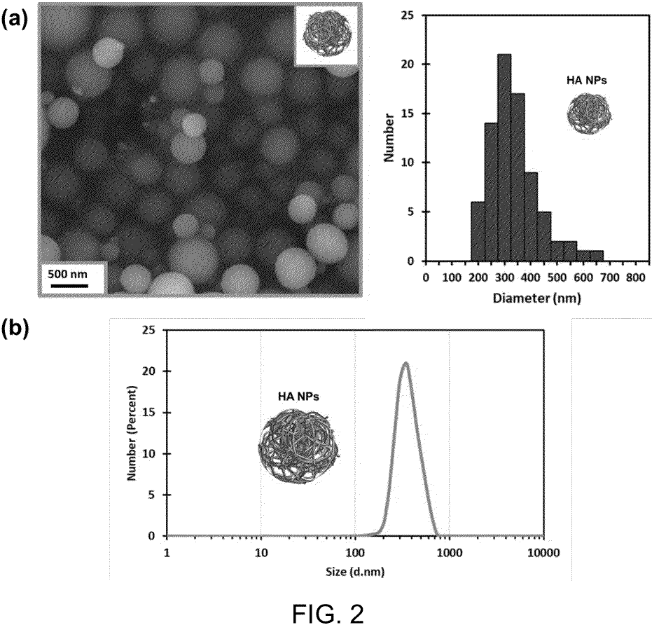

Successful interaction between chitosan chains and TPP was demonstrated by FT-IR spectroscopy (FIG. 2b). In the CS-TPP NPs sample, the stretching vibrations of O—H and N—H band became wider compared to those of native CS and TPP crosslinker, indicating an enhancement of the hydrogen bonding interactions. As well, the shift in the peak of —NH bending vibration from 1578 cm⁻¹ to 1637 cm⁻¹ was attributed to the interaction between the amino group of CS and the phosphate anion of TPP. Moreover, the appearance of a new peak at 1538 cm-1 assigned to N—O—P stretching vibration indicates that TPP anions were crosslinked successfully with the ammonium groups of CS to form CS-TPP NPs through ionotropic gelation method. A new shoulder peak at 1754 cm⁻¹ corresponding to the protonated acidic groups of HA (COO⁻) appeared in the HA NPs spectrum confirming the successful incorporation of HA with CS-TPP NPs, in addition to the presence of the characteristic peaks of CS and HA. The integration of AGP protein with HA NPs was assessed through the presence of all characteristic peaks of CS and AGP protein. Besides the reduction in the stretching frequency of all peaks due to the hydrogen bonding interaction between the protein and the NPs.

The zeta potential of the NPs was consistently reversed from + 31 ±0.5 mV to - 33 ±1 mV after the addition of HA, due to the accumulation of HA molecules on the surface of the NPs. A further change occurs in zeta potential value after the decoration of the NPs with AGP protein, where the zeta potential increased from - 33 ±1 mV to - 28 ±2 mV, as shown in FIG. 2a. This raise in zeta potential value could be explained by the partial shielding of the NP surface by AGP protein.

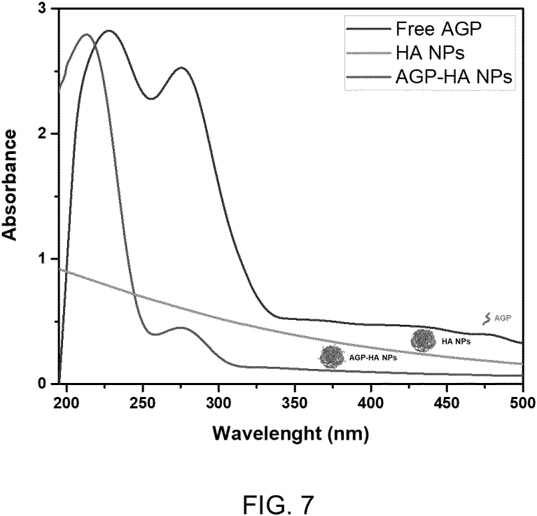

The UV-Vis spectroscopy exhibited a blue shift in the UV-Vis absorption of NPs decorated with AGP compared to free AGP, as shown in FIG. 7. This shift is indicated a presence of interaction between the NPs and AGP, which attributed to π→π∗ transitions in the peptide bonds of AGP, and thus verified the successful interaction between the NPs and AGP.

### In Vitro Studies

### AGP-HA NPs Inhibiting Migration and Invasion of MCF-7 Breast Cancer Cells

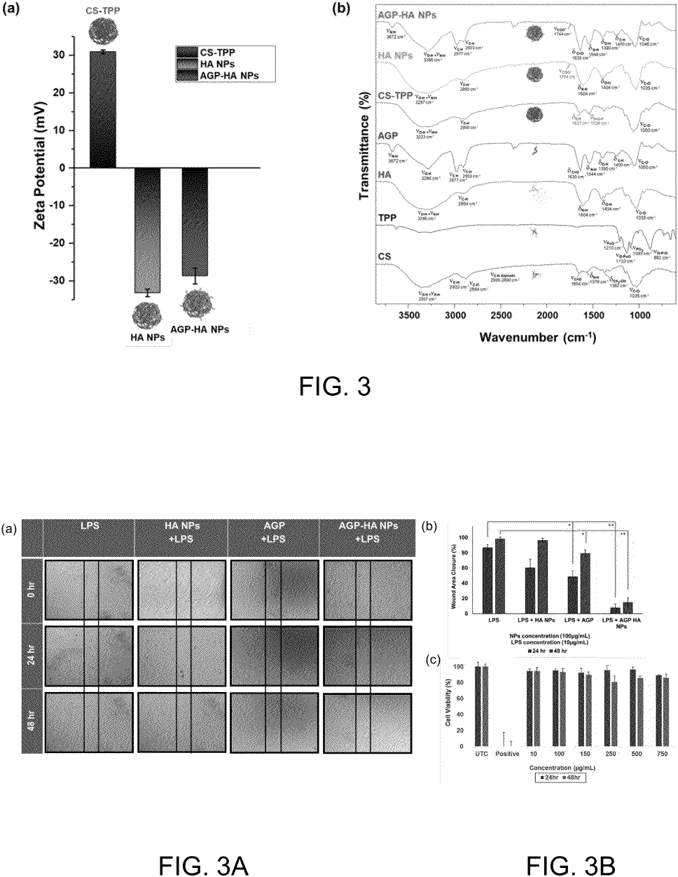

The biocompatibility of AGP-HA NPs in breast cancer cell line (MCF-7), was examined at 24 and 48 hr incubation time with different concentration. FIG. 3c, illustrates that NPs are biocompatible with significant cell survival at concentration of 100 µg/mL.

Cancer cell migration and invasion play a crucial role in cancer metastasis. Wound healing assays were performed on MCF-7 cell line to assess the efficiency of AGP-HA NPs in inhibiting the migratory ability of breast cancer cell.

To investigate the migratory ability of MCF-7cells, cells were incubated with 10 µg/mL LPS and 100 µg/mL of AGP-HA NPs, simultaneously, at 24 h and 48 h. LPS treatment has been known to be linked to tumor progression and metastasis in MCF-7 breast cancer cells.

As shown in FIGS. 3(a-b), AGP-HA NPs inhibited the migration dramatically, compared to the LPS- treated and LPS/AGP treated MCF-7 cells control groups. AGP-HA NPs significantly decreased migratory ability by 79±1% for 24 hr and 83±3% for 48 hr. Thus, indicating the ability of AGP-HA NPs in suppressing cells migration and invasion.

### LPS-Mediated Invasion and Migration of MDA-MB-231 Breast Cancer Cells

Pro-inflammatory factors and chemotactic cytokines are key players in mediating chemotherapeutic-resistance, cell proliferation, and metastasis in human cancers. Besides, lipopolysaccharide (LPS) is recognized as one of the most important promoters of cancer progression in many MDR mechanisms. LPS is known as a trigger and a master regulator of inflammatory responses in numerous cancer invasions or angiogenesis. It has been affirmed that LPS has a significant role in the stimulation of invasiveness and metastasis via a diversity of signal transduction pathways, particularly in MDA-MB-231 breast cancer cells.

In this study, the stimulation of inflammation-mimetic effect and the acquisition of chemoresistance in MDA-MB-231 cells were induced through constitutive activation using LPS, which persuades transient resistance to the cytotoxic drug (DOX).

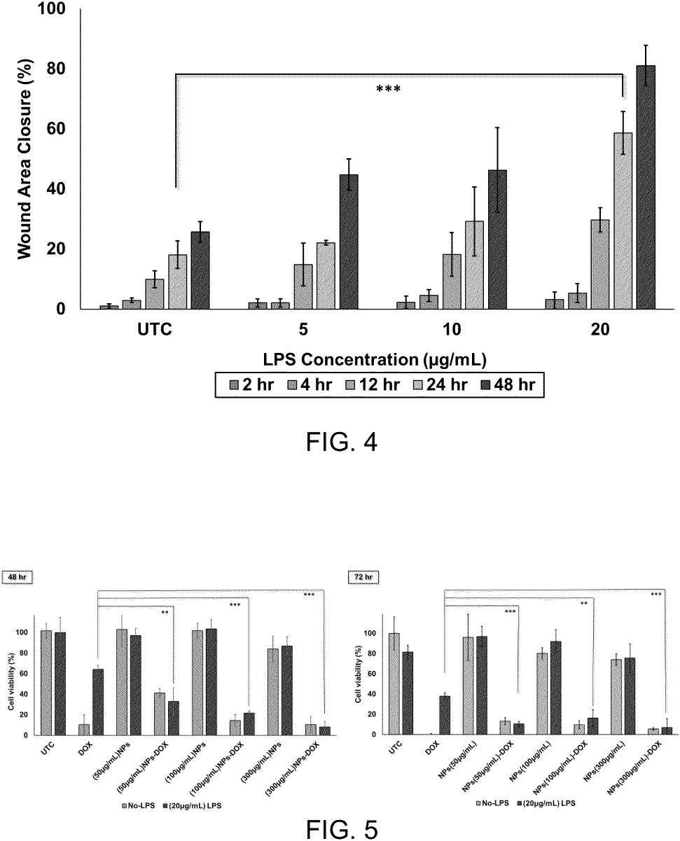

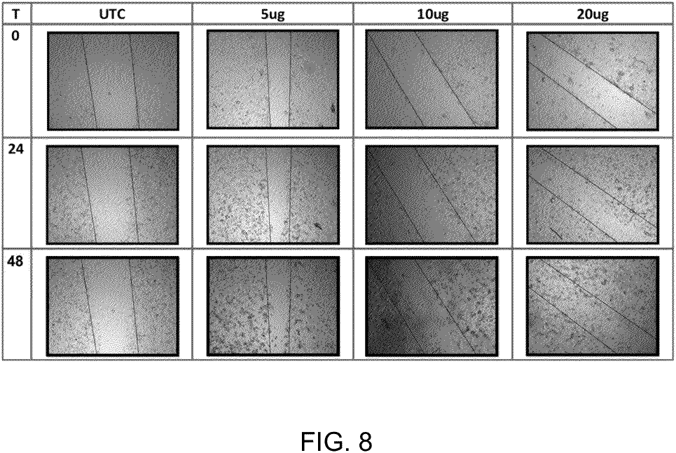

To evaluate how LPS could enhance the invasive potential of MDA-MB-231 cells, the Inventors used an in vitro wound-healing assay through monitoring and imaging the scratch closure from 0 to 48 hr intervals using Cytation 5™. Altered cell proliferation and migration were significantly noticeable in response to LPS exposure at a concentration of 20 µg/mL after 24 hr of incubation time in comparison to the untreated control, as shown in FIG. 4. On the contrary, higher doses of LPS-treatment at 30 and 50 µg/mL upon incubation for 24-48 hr drastically impaired the proliferation of MDA-MB-231. Administration of lethal doses of LPS (LD > 20 µg/mL) correlated to increased cytotoxicity in cells, which was attributed to an increased rate of apoptosis of tumor cells along with morphological alteration. As revealed in FIG. 5 and FIG. 8, LPS pretreated cells migrated three times faster than the untreated cells with p-value difference (p=0.0012).

These results confirm LPS capability to increase the invasiveness of aggressive breast cancer cells by about 41% at a concentration of 20 µg/mL.

### Modulation of DOX Resistance in Human Breast Cancer Cells via AGP-HA NPs

LPS ability to mediate DOX-resistance was examined to simulate the MDR phenotype of MDA-MB-231. Herein, there is compared the sensitivity of non-stimulated MDA-MB-231 cells and LPS-stimulated cells at an optimized migratory concentration of LPS (20 µg/mL) to DOX treatment. AlamarBlue cell viability assay was used to evaluate the ability of LPS activation to induce DOX resistance. Results showed that non-stimulated MDA-MB-231 cells were sensitive to DOX treatment at a concentration of 0.67 µg/mL since DOX reduced cell viability to 11±10% as shown in FIG. 5. On the contrary, LPS-stimulated cells were 64±4% viable after 24 hr of DOX-treatment. Remarkably, pretreatment with LPS for 24 hr was clearly able to reduce chemosensitivity, lessen DOX cytotoxicity, and increase cell survival approximately to 6-fold (p-value <0.0005) after 48 hr treatment. These quantitative results provide strong evidence of inflammation-based MDR mechanism specifically to DOX treatment in MDA-MB-231 cells. These results are consistent with previous reports of LPS-mediated resistance to the known MDR substrate DOX.

To investigate the influence AGP-HA NPs to modulate MDR, the Inventors first evaluated the cytotoxicity of AGP-HA NPs toward MDA-MB-231 cells at 48 and 72 hr of incubation. Notably, in FIG. 5 AGP-HA NPs showed high biocompatibility maintaining cell viabilities at 96 ±23% and 80 ±6% after incubation with MDA-MB-231 cells for 72 hr at concentrations of 50 and 100 µg/mL, respectively. Following that, the effect of AGP-HA NPs on restoring the sensitivity of cells to chemotherapeutic drugs was studied in response to the combined treatment of NPs and DOX in MDR phenotype cells using AlamarBlue cell viability assay.

The most potent cell growth-inhibitory effect was noticed in MDR-phenotype cells treated with NPs-DOX in-situ incubation compared with the free drug (FIG. 5). A direct relationship was observed between the concentration of NPs and cell sensitivity to DOX within certain periods of time (48 and 72 hr). Cell growth-inhibitory effect dramatically increased with increasing the concentration of NPs, demonstrating the restoration efficacy of NPs in overcoming MDR as shown in FIG. 5b.

Additionally, cell sensitivity and DOX cytotoxicity were enhanced prominently with increasing incubation time. For instance, after 48 hr of treatment, resistant cells incubated with 100 µg/mL of NPs were more sensitive to DOX in-situ treatment in comparison to the free drug, reducing cell viability dramatically from 64 ±4% to 21 ±2% (p-value<0.00002). Likewise, 72 hr-treatment resulted in enhanced inhibitory effects in a dose-dependent manner. This descriptive approach elucidated the indispensable role of AGP-decorated NPs to efficiently internalize DOX in MDR aggressive breast cancer cells.

### EXAMPLES

The following non-limiting examples, which are intended to be exemplary, further clarify the present disclosure

### Materials

Alpha-1 Acid Glycoprotein (AGP) CAT# (66455-27-4). PBS. High pure chitosan. Lipopolysaccharide (LPS) from E. coli 026:B6, Sigma (L8274-10MG). QuantiPro™ BCA Assay kit, Sigma, USA (QPBCA-1 KA). High purity chitosan (deacetylation degree over 75-85% mol and viscosimetric molecular weight 50-190 kDa), sodium triphosphate pentabasic (TPP), 1 M hydrochloric acid (HCl), and sodium hydroxide (NaOH) were obtained from Sigma-Aldrich (UK). Milli-Q water was used in all synthetic experiments. Hyaluronic acid (HA; weight average molecular weight ≈ 200 kDa) was purchased from Medipol SA (Switzerland). Regenerated cellulose (RC) dialysis membrane (MWCO 1000 kDa) was obtained from SpectraP or, Spectrum Laboratories Inc. (USA).

### Methods

Dynamic light scattering (DLS). Hydrodynamic diameter (Z-average size), polydispersity index (PDI), and zeta potential measurements were always performed at room temperature using a Zetasizer Nano ZS instrument (Model ZEN3600, Malvern Instruments Ltd., UK) fitted out with a solid-state HeNe laser (λ = 633 nm) at a scattering angle of 173°. Scanning electron microscopy (SEM) was recorded by JSM-IT500HR InTouchScope. Nano Spray Dryer (Model B-90, BÜCHI Labortechnik AG, Flawil, Switzerland) was used a dryer. Millipore Labscale TFF System (Millipore Corporation, MA) was used, along with a Pellicon XL TFF cassette with a 500 kDa molecular weight cut-off membrane (Millipore Corporation, MA), as an ultrafiltration technique. FTIR spectra were recorded using a Thermo Scientific spectrometer (Nicolet iS10). Absorption spectra were recorded using a Varian Cary 5000 spectrophotometer. Powder XRD measurements were performed using a Panalytical X′Pert Pro X-ray powder diffractometer using the Cu Kα radiation (40 V, 40 mA, λ = 1.54056 Å) in a θ - θ mode from 20 ° to 90 ° (2 θ).

### NANOPARTICLES SYNTHESIS

### Synthesis of CS-TPP NPs

A 0.069% wt. chitosan solution was prepared by dissolving CS (69 mg) in 4.6 mM HCl solution (100 mL) and kept stirring overnight at room temperature. The pH was then adjusted to 5 by adding appropriate volumes of 0.5 M NaOH. A 0.1% wt. TPP solution was prepared by dissolving TPP (10 mg) in deionized water (10 mL) and stirred for 30 min at room temperature, then the pH of the solution was adjusted to 5 using 0.5 M HCl. Both solutions were filtered through a 0.22 µm pore size filter. 7 mL of TPP solution was added to CS solution (93 mL) for a final volume of 100 mL, making a 9:1 CS: TPP mass ratio to produce 0.064 and 0.0071 wt.% of CS and TPP, respectively. Then, under magnetic stirring and agitation, complexation to form the CS NPs was carried for 30 min at room temperature. Finally, the dispersed NPs were dialyzed against deionized water (2 h, MWCO 1000 kDa).

### Synthesis of CS-TPP-HA NPs

A 0.2% wt. HA solution was prepared by dissolving HA (120 mg) in deionized water (60 mL) and kept stirring overnight at room temperature. Then the solution was filtered through a 0.22 µm pore size filter.

50 mL of CS-TPP NPs solution was slowly added under vigorous stirring to an equal volume of HA solution and stirred for 30 min at room temperature. The dispersed NPs were then dialyzed against deionized water (2 h; MWCO 1000 kDa). The produced NPs were then dried using Buchi’s Nano Spray Dryer in the open-loop configuration, where inlet temperature was adjusted to 120° C., and both, motor and spray power, were set to 50%.

### Synthesis of CS-TPP-HA-AGP NPs

A 0.1% wt. AGP solution was prepared by dissolving AGP (1 mg) in deionized water (1 mL). The volume ratio of NPs: AGP (1:3) was prepared with a final volume of 300 µL. Where 225 µL of AGP (1 mg/mL) solution was added to 75 µL of HA-CS NPs solution (1 mg/mL) for a final volume of 300 µL making a 1:3 NPs: AGP volume ratio. And, then kept with gentle stirring for 24 h at room temperature. The sample was then filtered using Millipore’s Labscale TFF System to remove unbounded AGP.

### In Vitro Study

### Cell Culturing

MDA-MB231 cells were obtained from the American Type Culture Collection (ATCC-CCL-61, USA) and subsequently cultured in Dulbecco’s modified Eagle’s medium (DMEM) supplemented with 10% FBS and 1% penicillin/streptomycin solution at 5% CO2 and 37° C. At 85% confluency, cells were detached enzymatically using 0.25% trypsin and cultured in 96 multi-well plates with a seeding density of 6×103 cells per well. Next, MDA-MB231 cells were incubated for 24 hr, after which they were treated with different concentrations (50, 100 and 300 µg/mL) of CS-TPP-HA-AGP NPs for 48/72 hr. Cells not being exposed to CS-TPP-HA-AGP nanoparticles served as untreated control (UTC).

### Cell Viability Assay

AlamarBlue assay was conducted to investigate the % viability of MDA-MB231 cells after being exposed to a range of concentrations (50, 100 and 300 µg/mL) of CS-TPP-HA-AGP NPs incubated for 48/72 hr. As incubation completed, culture media were replaced with fresh media containing 10% of AlamarBlue reagent and incubated for 3-4 hr at 5% CO2 and 37° C. The UTC served as a negative control. Fluorescence spectra were recorded at λex:550, λem:600 using BioTek Cytation 5™ multi-mode Microplate Reader-Gen5™ software.

### LPS Enhances the Invasive Potential in MDA-MB-231

In this study, the mechanism of chemoresistance in human triple-negative breast cancer cells (MDA-MB-231) induced by exposure to lipopolysaccharide (LPS). LPS was suggested to promote cancer cell migration. In in vitro scratch assays, the cells were seeded in 12-well plates at a density of 6×10⁵ cells/ well. After incubation overnight, cells were stimulated with LPS (10, 20, 30, and µg/ml) for 24 hr, and then scraped by a p20 pipette tips to create a straight-line cell-free scratch. Each well was washed with PBS three times to remove the remaining unattached cells and debris. The scratch area was marked and photographed in different time intervals (0, 4, 12, 24, and 48) hr. The distances were measured by the software ImageJ, and cell motility was quantified by measuring the distance between the migrating cell boundaries. Data were analyzed statistically.

### LPS-Mediates DOX-Resistance Activation

MDA-MB-231 cells were plated out in 96-well tissue culture plates at 1 ×10⁴ cells per well and treated with or without LPS at concentration of (20 µg/mL) for 24 hr. Cells were then left untreated or treated simultaneously with (0.67 µg/mL) of the chemotherapeutic drug (DOX) and various doses of CS-TPP-HA-AGP NPs (50, 100, and 300 µg/ml). After further incubation for 48 or 72 hr, Cells were subjected to AlamarBlue cell viability assay. As incubation completed, culture media were replaced with fresh media containing 10% of AlamarBlue reagent and incubated for 3-4 hr at 5% CO₂ and 37° C. The UTC served as a negative control and LPS-stimulated/non-stimulated control groups were untreated with nanoparticles. Fluorescence spectra were recorded at λex:550, λem:600 using BioTek Cytation 5™ multi-mode Microplate Reader-Gen5™ software.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Other embodiments of the invention will be apparent to those skilled in the art from consideration of the specification and practice of the invention disclosed herein. It is intended that the specification and examples be considered as exemplary only, with the true scope of the invention being indicated by the following claims.

While the present disclosure has been shown and described with reference to particular embodiments thereof, it will be understood that the present disclosure can be practiced, without modification, in other environments. The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments.

Computer programs based on the written description and disclosed methods are within the skill of an experienced developer. Various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A composition for reducing multi-drug resistance of tumor cells to chemotherapeutic drug molecules, the composition comprising:
at least one nanoparticle comprising at least one anti-inflammatory protein, wherein the at least one anti-inflammatory protein comprises an alpha-1 acid glycoprotein (AGP), wherein the composition inhibits the proliferation and the migration of tumor cells by suppressing overexpressed pro-inflammatory cytokines.

2. The composition of claim 1, wherein the at least one nanoparticle comprises hyaluronic acid-chitosan.

3. The composition of claim 1, wherein the at least one nanoparticle has a size distribution ranging between 200 and 500 nm.

4. A method of making a composition comprising at least one nanoparticle comprising at least one anti-inflammatory protein, wherein the at least one anti-inflammatory protein comprises an alpha-1 acid glycoprotein (AGP),
the method comprising sequential ionic gelation followed by at least one spray drying step.

5. The method of claim 4, wherein the sequential ionic gelation steps comprise:
preparing chitosan tripolyphosphate (CS/TPP) nanoparticles by using inter-molecular and intra-molecular ionic linkages created between negatively charged groups on the pentasodium tripolyphosphate (TPP), and positively charged amino groups of the chitosan.

6. The method of claim 5, further comprising adding the CS-TPP nanoparticles to a hyaluronic acid solution to produce hyaluronic acid nanoparticles.

7. The method of claim 6, wherein the hyaluronic acid nanoparticles are configured such that hydrogen bonding and electrostatic attractions occur between the carboxylic groups of hyaluronic acid and amine groups of chitosan.

8. The method of claim 5, the at least one spray drying step produces a powder comprising said nanoparticles.

9. The method of claim 8, further comprising mixing the powder comprising said nanoparticles with AGP to produce hyaluronic acid-chitosan nanoparticles decorated with AGP.

10. A method of treating cancer, comprising:
treating cancer cells with a composition comprising at least one nanoparticle comprising at least one anti-inflammatory protein, wherein the at least one anti-inflammatory protein comprises an alpha-1 acid glycoprotein (AGP), and
treating said cancer cells with an anti-cancer, chemotherapeutic drug.

11. The method of claim 10, where treating cancer cells with a composition comprising at least one nanoparticle comprising at least one anti-inflammatory protein, wherein the at least one anti-inflammatory protein comprises an alpha-1 acid glycoprotein (AGP) is done prior to or simultaneous with treating said cancer cells with an anti-cancer, chemotherapeutic drug.

12. The method of claim 10, wherein the at least one nanoparticle comprises hyaluronic acid-chitosan.

13. The method of claim 10, wherein the step of treating cancer cells with said composition comprises using an amount of said composition sufficient to sensitize said cancer cells prior to exposing said cancer cells to the anti-cancer, chemotherapeutic drug.

14. The method of claim 10, wherein said cancer comprises metastatic cancer.

15. The method of claim 10, wherein said cancer comprises metastatic breast cancer.
